SEQUENCE LISTING (14 sequences)

SEQ ID NO: 1 - protein, 115 aa, Porcine circovirus 2
PSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQR

SEQ ID NO: 2 - protein, 115 aa, synthetic construct
PSKKNGRSGPQPHKRWVFTLNNPSEDERKKIRDLPISLFDYFIVGEEGNEEGRTPHLQGFANFVKKQTFNKVKWYLGARVWLQPAKGTDQQNKEYCSKEGNLLMECGAPRSQGQR

SEQ ID NO: 3 - protein, 115 aa, synthetic construct
PSKKNGRSGPQPHKRWVFTLNNPSEDERKKIRDLPISLFDYFIVGEEGNEEGRTPHLQGFANFVKKQTFNKVKWYLGARIWTQPAYGTDQQNKEYCSKEGNLLMECGAPRSQGQR

SEQ ID NO: 4 - protein, 115 aa, synthetic construct
PSKKNGRSGPQPHKRWVFTLNNPSEDERKKIRDLPISLFDYFIVGEEIGPALRTPHLQGFANFVKKQTFNKVKWYLGARIWTQPAYGTDQQNKEYCSKEGNLLMECGAPRSQGQR

SEQ ID NO: 5 - protein, 7 aa, synthetic construct
CHIEKAK

SEQ ID NO: 6 - protein, 12 aa, synthetic construct
GARVWLQPAKGT

SEQ ID NO: 7 - protein, 12 aa, synthetic construct
GARIWTQPAYGT

SEQ ID NO: 8 - protein, 12 aa, synthetic construct
GARIQTQPAYGT

SEQ ID NO: 9 - protein, 12 aa, synthetic construct
  VARIANT 4: note = X=I or V
  VARIANT 6: note = X= T or L
  VARIANT 10: note = X= Y or K
GARXWXQPAXGT

SEQ ID NO: 10 - protein, 5 aa, synthetic construct
IGPAL

SEQ ID NO: 11 - RNA, 17 nt, synthetic construct
  source 1..17: mol_type = other RNA
cgtaaaatattaccgtc

SEQ ID NO: 12 - DNA, 17 bp, synthetic construct
  misc_feature 1..4: note = DNA
  misc_feature 5..14: note = RNA
  misc_feature 15..17: note = DNA
  source 1..17: mol_type = other DNA
cgtaaaatattaccgtc

SEQ ID NO: 13 - DNA, 15 bp, synthetic construct
  source 1..15: mol_type = other DNA
aagtattaccagaaa

SEQ ID NO: 14 - protein, 292 aa, Duck circovirus
MAKSGNYSYKRWVFTINNPTFEDYVHVLEFCTLDNCKFAIVGEEKGANGTPHLQGFLNLRSNARAAALEESLGGRAWLSRARGSDEDNEEYCAKESTYLRVGEPVSKGRSSDLAEATSAVMAGVPLTEVARKFPTTYVIFGRGLERLRHLIVETQRDWKTEVIVLIGPPGTGKSRYAFEFPAENKYYKPRGKWWDGYSGNDVVVMDDFYGWLPYDDLLRITDRYPLRVEFKGGMTQFVAKTLIITSNREPRDWYKSEFDLSALYRRINKYLVYNIDKYEPAQACTLPFPINY

(19) United States
(12) Patent Application Publication
(10) Pub. No.: US 2026/0286325 A1
(43) Pub. Date: Sep. 24, 2026

(54) COMPOSITIONS AND METHODS INCLUDING RNA-BINDING PROTEINS

(71) Applicant: REGENTS OF THE UNIVERSITY OF MINNESOTA, Minneapolis, MN (US)

(72) Inventors: Wendy Ryan Gordon, St. Paul, MN (US); Adam Trey Smiley, Minneapolis, MN (US); Kassidy J. Tompkins, Minneapolis, MN (US)

(21) Appl. No.: 19/473,912

(22) PCT Filed: Apr. 10, 2024
(86) PCT No.: PCT/US2024/023882

§ 371 Date: Oct. 9, 2025

Related U.S. Application Data
(60) Provisional application No. 63/458,358, filed on Apr. 10, 2023.

(51) Int. Cl. C12N 9/22; C12N 7/00; C12N 9/78

(52) U.S. Cl. CPC C12N 9/22; C12N 7/00; C12N 9/78; C07K 2319/00; C12N 2750/10022; C12Y 305/04004

(57) ABSTRACT

An RNA-binding protein includes a substitution mutation to a semi-conserved domain, said semi-conserved domain including the amino acid sequence of SEQ ID NO:5. 21. The RNA-binding protein may be an HUH endonuclease, such as a virally-derived HUH endonuclease, such as PCV2. A fusion protein includes an RNA-binding protein and a protein involved in gene editing. A composition includes an RNA-binding protein or a fusion protein including an RNA-binding protein.

### CROSS-REFERENCE TO RELATED APPLICATION

This application claims the benefit of U.S. Provisional Patent Application No. 63/458,358 filed Apr. 10, 2023, which is incorporated herein by reference in its entirety.

### GOVERNMENT FUNDING

This invention was made with government support under GM119483 awarded by the National Institutes of Health. The government has certain rights in the invention.

### SEQUENCE LISTING

This application contains a Sequence Listing electronically submitted via Patent Center to the United States Patent and Trademark Office as an XML file entitled “0110000703WO01” having a size of 16,078 bytes and created on Mar. 29, 2024. The information contained in the Sequence Listing is incorporated by reference herein.

### SUMMARY

This disclosure describes, in one aspect, an RNA-binding protein having a substitution mutation to a semi-conserved domain, said semi-conserved domain having the amino acid sequence of SEQ ID NO:5.

In one or more embodiments, the RNA-binding protein includes a catalytic tyrosine residue and one or two metal-coordinating histidine residues. In one or more embodiments, the RNA-binding protein is an HUH endonuclease.

In one or more embodiments, the RNA-binding protein is virally derived. In one or more embodiments, the RNA-binding protein is derived from a virus belonging to the genus Circovirus. In one or more of these certain embodiments, the RNA-binding protein derived from a porcine circovirus (PCV), such as porcine circovirus-2 (PCV2).

In one or more embodiments, the RNA-binding protein includes an amino acid sequence having at least 70% identity to SEQ ID NO:1. In one or more of these certain embodiments, the RNA-binding protein has a substitution mutation to the position functionally equivalent to position 81 in SEQ ID NO:1 and/or a substitution mutation to the position functionally equivalent to position 86 in SEQ ID NO:1. The substitution mutation to position 81 and/or the substitution mutation to position 86 may be mutation to an aromatic amino acid. In one or more of these embodiments, the substitution mutation to position 81 may be a mutation to tryptophan and/or the mutation to position 86 may be a mutation to tyrosine.

In one or more embodiments, the RNA-binding protein includes the amino acid sequence of any one of SEQ ID NO:2, SEQ ID NO:3, or SEQ ID NO:4.

In one or more embodiments, the RNA-binding protein includes a divalent cation, such as Mg²⁺ or Mn²⁺.

In another aspect, the present disclosure describes a nucleic acid expressing an RNA-binding protein consistent with those described herein.

In another aspect, the present disclosure describes a composition having an RNA-binding protein consistent with those described herein. The composition may include Mn²⁺. The composition may include the on sequence of the RNA-binding protein. The composition may include a nucleic acid, wherein the nucleic acid includes (e.g., encodes) the on sequence of the RNA-binding protein.

In another aspect, the present disclosure describes a fusion protein having the RNA-binding protein consistent with those described herein. In one or more embodiments, the fusion protein includes a protein involved in gene editing. In one or more embodiments, the fusion protein may include an adenosine deaminase, such as an adenosine deaminase acting on RNA (ADAR).

In another aspect, the present disclosure describes a composition having a fusion protein consistent with those described herein. The composition may include Mn²⁺. The composition may include the on sequence of the RNA-binding protein. The composition may include a nucleic acid, wherein the nucleic acid includes (e.g., encodes) the on sequence of the RNA-binding protein.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Overview of HUH endonuclease structure and function. (A) Graphical depiction of the hypothesized mechanism of protein-RNA covalent linkage. The HUH-endonuclease recognizes its specific sequence, cleaves it, and remains covalently bound to the substrate's newly exposed 5′ end through a phospho-tyrosine covalent linkage. (B) SDS-PAGE gel analysis of HUH-RNA reactions in the presence of a variety of additives. Higher molecular weight bands are indicative of a protein-RNA covalent linkage. The addition of the RNA substrate including a nuclease-specific sequence enables covalent linkage. This adduct can be truncated by the addition of RNase and is unaffected by the addition of DNase, indicating that this is a protein-RNA covalent linkage.

FIG. 2. Graphical representation of the engineering/directed evolution scheme for the selection of engineered HUH endonucleases with enhanced reaction efficiency towards non-cognate RNA substrates.

FIG. 3. Activity, structure, and sequence of mutated variants E1, E2, and E3. (A) Comparative molecular beacon cleavage assays depicting the difference in reaction efficiency on a non-cognate Q/F RNA substrate throughout the engineering process. (B) The co-crystal structure of the PCV2 HUH-endonuclease in complex with its cognate DNA structures. Mutagenized portions of the protein with respect to wild type are colored according to their respective engineered variants (mutations are progressive, meaning E3 contains all mutations from E1 and E2). (C) Multiple sequence alignment of the wildtype (WT, SEQ ID NO:1) and engineered variants (E1, SEQ ID NO:2; E2, SEQ ID NO:3; E3, SEQ ID NO:4) with mutagenized residues colored to match respective engineered variants.

FIG. 4. In vitro HUH cleavage reactions under restrictive conditions to compare reaction efficiency under less saturated conditions between the WT, E1, and E2.

FIG. 5. Quantification of activity of engineered HUH endonuclease E1 and E2. (A) Flow cytometry plots of populations of yeast cells surface-expressing either wild-type PCV2, or heterogenous populations of yeast expressing engineered PCV2 variants post the stated round of flow cytometry selection. Mutagenic libraries were selected via subsequent rounds of magnetic activated cell sorting (MACS) and fluorescence activated cell sorting (FACS), ultimately producing the populations of the most efficient group of mutants shown in the FACS1 and FACS2 populations. Next-generation sequencing of these populations identified the most functional variants.

FIG. 6. HUH-ADAR RNA-editing experiments. (A) Graphical depiction of the HUH-ADAR system and its putative mechanism. (B) Flow cytometry histograms comparing the fluorescence of non-transfected reporter cells to those transfected with a plasmid that expresses the E3 HUH-ADAR RNA editing system and a guiding RNA after 24 hours. (C) Bar graph comparing the percentage of fluorescent cells across multiple RNA-editing treatment conditions.

FIG. 7. Bar graph comparing the percentage of fluorescent cells across multiple RNA-editing treatment conditions. These conditions employed chemically stabilized versions of guiding RNAs containing the nuclease's specific sequence delivered 24 hours post HUH-ADAR plasmid transfection to overcome cellular RNases, including methylated RNA bases and phosphorothiorated backbones.

FIG. 8. Schematic representations of yeast display and HUH reactions. (A) Graphical depiction of the yeast surface display-based protein engineering strategy used to engineer this enzyme. (B) Cartoon representation of an HUH reaction with ssRNA at 37° C. with Mn²⁺.

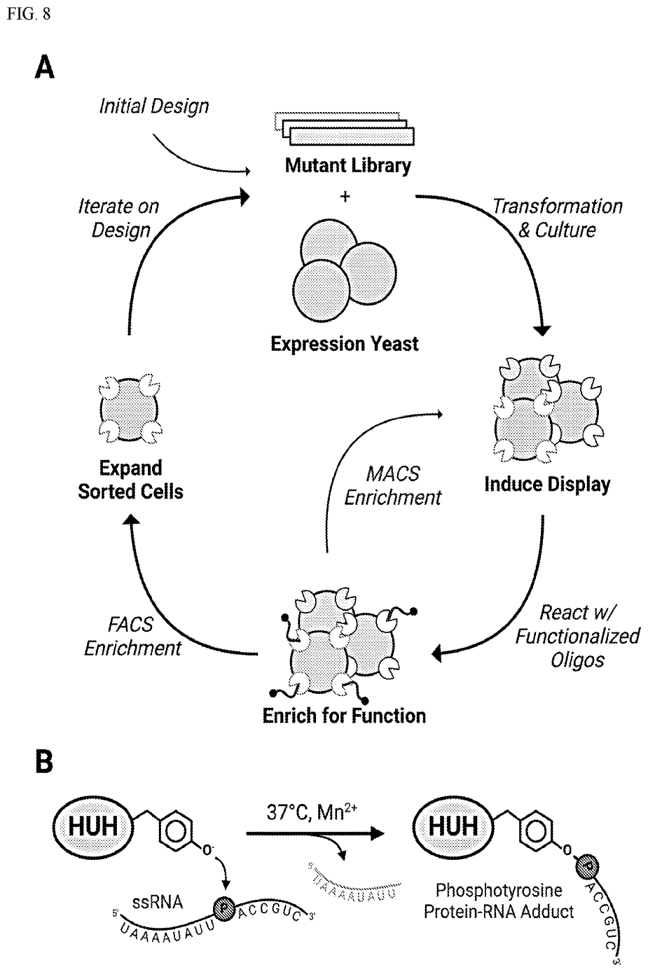

FIG. 9. Flow cytometric analysis of yeast cells expressing PCV2 (top right), a heterogenous population of different engineered enzymes after two rounds of MACS and one round of FACS selection on the first library (bottom left), and a heterogenous population of different engineered enzymes after two rounds of MACS and one round of FACS selection on the second library (bottom right). The sequences beneath the plots are from NGS data collected post-sort and represent the proteins analyzed in the plots above the sequence. The sequences above the sequence logos represent the consensus sequence prior to engineering. That is, the consensus sequences prior to FACS1 is the WT sequence, and the consensus sequence prior to FACS2 is an inferred consensus sequences from the NGS data from FACS1 where highly ambiguous residues (like positions 80, 82, and 83) are indicated by X.

FIG. 10. Structural and substrate differences of PCV2 WT, PCV2 E1, and PCV2 E2. (A) MSA of the mutagenized section in the WT (SEQ ID NO:1), E1 (SEQ ID NO:2), and E2 (SEQ ID NO:3) variants of the enzyme with a crystal structure showing which residues they are in relation to the three-dimensional structure. (B) Covalent linkage plots for DNA (left) and RNA (right) for the PCV2 WT, PCV2 E1, and PCV2 E2 variants.

FIG. 11. Cleavage plots for DNA (left) and RNA (right) for the WT, E1, and E2 variants. Each enzyme was reacted with a ‘molecular beacon’ that has a fluorophore on one end and a quencher on the other. Affinity data for DNA (left) and RNA (right) for the WT, E1, and E2 variants. Data was collected using fluorescence polarization (n=12 for each).

FIG. 12. Covalent linkage plots for DNA and RNA of the E2 protein including H81, K86, or both on the PCV2 E2 backbone. H81 with or without K86 effectively ablated cleavage of RNA by the enzyme.

### DETAILED DESCRIPTION OF ILLUSTRATIVE EMBODIMENTS

The ability to covalently attach RNA to proteins has broad applications in, for example, gene editing. While many proteins are known to cleave or otherwise transiently act on RNA, fewer proteins are known to form stable, covalent bonds to RNA. As used herein, an “RNA-binding protein” (or “RNA binding protein”) forms a covalent bond to RNA, sometimes in a sequence-specific manner. In one aspect, the present disclosure is directed to an RNA-binding protein, wherein the RNA-binding protein forms a covalent attachment to a single-stranded RNA (ssRNA) molecule. An RNA-binding protein consistent with the compositions and methods of the present disclosure includes an amino acid sequence and may be identified by an amino acid motif.

### RNA-Binding Proteins

In one or more embodiments, an RNA-binding protein of the present disclosure is an HUH endonuclease. HUH endonucleases are enzymes that cleave and covalently attach to a single-stranded DNA (ssDNA) substrate in a sequence specific manner. These endonucleases are characterized by an active site including a pair of histidine (H) residues separated by a bulky hydrophobic residue (U). An HUH motif may alternately include one histidine, a bulky hydrophobic residue, and a glutamine used for cation coordination in place of the second histidine. Cation coordination triads are completed by a third residue, often glutamic acid or another histidine. This trio or pair of amino acids can coordinate a divalent cation, such as magnesium or manganese. When an HUH endonuclease binds a ssDNA substrate, the cation polarizes the ssDNA phosphate backbone, allowing the catalytic tyrosine to attack, cleave, and form a covalent linkage to the newly exposed 5′ end.

In their natural context, HUH endonucleases often play a role in DNA replication, such as rolling circle replication, or create a nick in plasmid DNA to facilitate mobilization, e.g., of a bacterium. The sequence of ssDNA substrate bound by an HUH endonuclease is often referred to as its “ori” sequence. Typical ori sequences are between 10 nucleotides and 40 nucleotides in length. One example of an ori sequence is shown in SEQ ID NO:13.

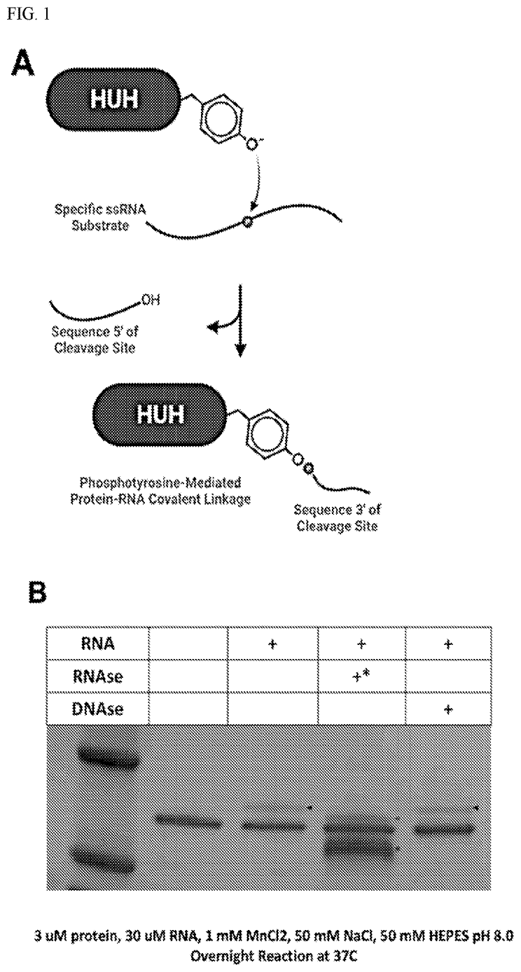

ssDNA-binding HUH endonucleases have been used for numerous biotechnology applications to covalently link a protein of interest to a ssDNA of interest under physiological conditions. Thus far, HUH endonuclease use has been limited to ssDNA binding. Surprisingly, several HUH endonucleases are shown herein to bind ssRNA. This reaction may follow a similar reaction mechanism to that of an HUH endonuclease reacting with ssDNA (FIG. 1A). However, the naturally occurring HUH endonucleases identified thus far react less efficiently with ssRNA than with ssDNA. Thus, improvements to HUH endonucleases to increase the efficiency of reaction with ssRNA are presented herein.

In one aspect, an RNA-binding protein of the present disclosure is an HUH endonuclease including one or more mutations. The one or more mutations typically increase the reaction efficiency of the RNA-binding protein with ssRNA. Multiple mutations are identified herein.

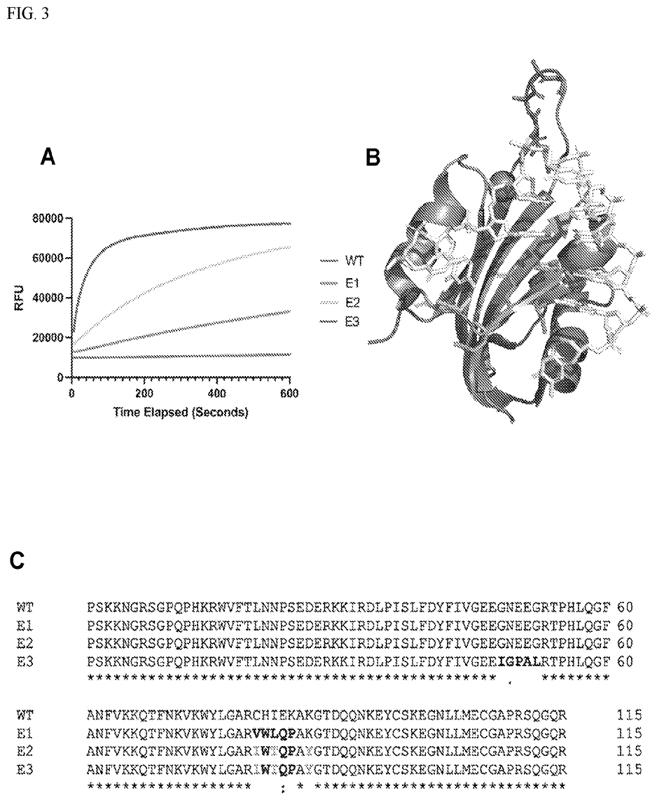

Proteins of the present disclosure include an HUH motif, characterized as an active site including a cation-coordinating histidine (H) and glutamine (Q) residue pair separated by a bulky hydrophobic residue (U), often leucine. An HUH motif may alternately include the more typical pair of histidine (H) separated by a bulky hydrophobic residue (U). The amino acids of an HUH motif may be adjacent within the secondary structure of a protein, or they may be separate within the secondary structure of a protein. The amino acids of an HUH motif are typically proximal to each other within the tertiary structure of a protein. Examples of proteins including HUH motifs include, for example, SEQ ID NO:1, which includes a two-amino acid HUH motif including amino acid residues 56 (H), 57 (L), and 58 (Q), and the catalytic motif amino acid residue 95 (Y). FIG. 3B depicts the tertiary structure of a protein having the amino acid sequence of SEQ ID NO:1.

In one or more embodiments, an RNA-binding protein of the present disclosure may be derived from a bacterium or a virus. As used herein, a protein “derived” from a source includes all or part of the amino acid sequence of a protein that naturally occurs in that source. In one or more embodiments, the RNA-binding protein may be derived from a virus belonging to the family Geminiviradae, such as wheat dwarf virus. In one or more embodiments, the protein may be derived from a virus belonging to the genus Circovirus, such as porcine circovirus (PCV) 1-4 or duck circovirus. In one or more embodiments, the protein may be derived from a virus belonging to the family Nanoviradae, such as Fababean Necrotic Yellow Virus. Suitable proteins derived from viruses that include an HUH domain include, but are not limited to, porcine circovirus-2 N-terminal replicase (PCV2, sometimes referred to as “PCV2 WT,” “WT PCV2,” and “PCV2 WT,” SEQ ID NO:1), and duck circovirus protein (DCV, SEQ ID NO:14). Additional HUH endonucleases are described in U.S. Pat. No. 10,717,773, which is incorporated by reference herein.

In one or more embodiments, an RNA-binding protein of the present disclosure may have at least 60%, at least 70%, at least 75%, at least 80%, at least 85%, at least 90%, at least 92%, at least 94%, at least 95%, at least 96%, at least 97%, at least 98%, at least 99%, or 100% sequence identity to a sequence disclosed herein. An RNA-binding protein of the present disclosure may be structurally similar to an RNA-binding protein disclosed herein. As used herein, an engineered RNA-binding protein, such as a mutated RNA-binding protein, may be “structurally similar” to a wild-type RNA-binding protein if the amino acid sequence of the engineered RNA-binding protein possesses a specified amount of sequence similarity and/or sequence identity compared to the wild-type RNA-binding protein.

Structural similarity of two amino acid sequences can be determined by aligning the residues of the two sequences (for example, a candidate RNA-binding protein and a reference, or wild-type, RNA-binding protein described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate RNA-binding protein is the RNA-binding protein being compared to the reference RNA-binding protein. A candidate RNA-binding protein that has structural similarity with a reference RNA-binding protein and RNA-binding protein activity is a mutated RNA-binding protein. As used herein, a “mutated” protein includes one or more amino acids that do not naturally occur at a certain position of the protein and/or lacks one or more amino acids that naturally occur at a certain position in the protein. A mutated protein may include a deletion of one or more amino acids, an insertion of one or more amino acids, or a substitution mutation of one or more amino acids. A “substitution mutation” as used herein is the replacement of one amino acid within a sequence with any other amino acid. Typically, an amino acid is mutated to a naturally occurring amino acid, however, non-natural amino acids are contemplated.

In one or more embodiments, an RNA-binding protein of the present disclosure is a mutated protein. The mutated RNA-binding protein may include at least one, at least two, at least three, at least four, at least five, at least six, at least seven, at least eight, at least nine, or at least ten mutated amino acid residues relative to a wild-type RNA-binding protein.

Described herein are particular amino acids of an RNA-binding protein which may be mutated to increase RNA-binding ability. Amino acids of particular interest may be identified in any manner. As described herein, a library-based approach may be used to identify amino acids of interest. In one or more embodiments, a yeast display approach may be used to identify RNA binding proteins of interest.

In one or more embodiments, an RNA-binding protein of the present disclosure includes an amino acid sequence including a substitution mutation, An RNA-binding protein of the present disclosure may include one or more substitution mutations to amino acids at positions functionally equivalent to amino acids 48-52 of SEQ ID NO:1. An RNA-binding protein of the present disclosure may include one or more substitution mutations to amino acids at positions functionally equivalent to amino acids 80-86 of SEQ ID NO:1.

In one or more particular embodiments, an RNA-binding protein of the present disclosure includes an amino acid sequence including a substitution mutation to the positions functionally equivalent to amino acid 81 and amino acid 86 of SEQ ID NO:1. Amino acid 81 of SEQ ID NO:1 is a histidine (H), thus, amino acid 81 may be mutated to any amino acid other than histidine. In one or more embodiments, amino acid 81 of SEQ ID NO:1 is mutated to an aromatic amino acid, such as tryptophan (W). Amino acid 86 of SEQ ID NO:1 is a lysine (K), thus, amino acid 86 may be mutated to any amino acid other than lysine. In one or more embodiments, amino acid 86 of SEQ ID NO:1 is mutated to an aromatic amino acid, such as tyrosine (Y). A protein of the present disclosure may include a substitution mutation of an amino acid equivalent to amino acid 81 of SEQ ID NO:1 to tryptophan, a substitution mutation of an amino acid equivalent to amino acid 86 of SEQ ID NO:1 to tyrosine, or both.

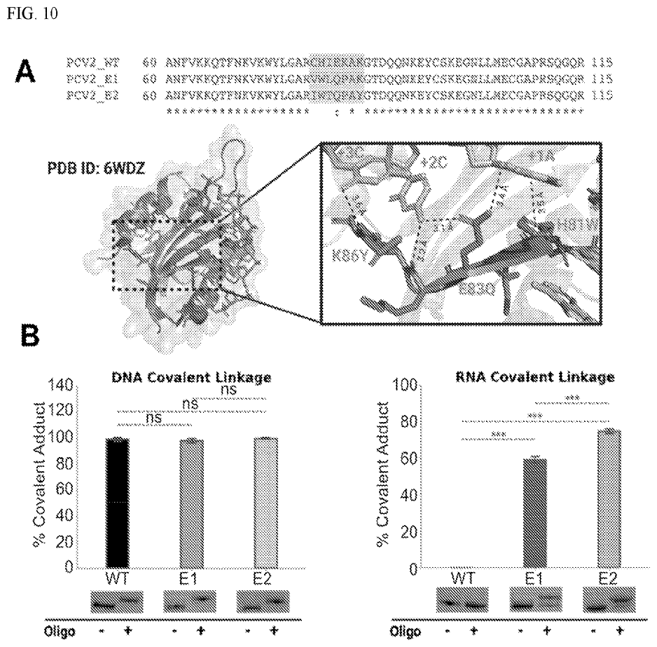

In one or more embodiments, an RNA-binding protein of the present disclosure includes the amino acid sequence of E1 PCV2 (sometimes referred to as “PCV2_E1,” “E1,” or SEQ ID NO:2). In one or more embodiments, an RNA-binding protein of the present disclosure includes an amino acid sequence having at least 70%, at least 75%, at least 80%, at least 85%, at least 90%, at least 95%, at least 98%, or at least 99% sequence identity with SEQ ID NO:2. As is described herein, E1 PCV2 is at least partially characterized by a sequence spanning from V80 to K86 or G77 to T88 (Motif A, SEQ ID NO:6). As described in the Examples of the present disclosure, E1 PCV2 exhibits increased reactivity with RNA relative to WT PCV2 (FIG. 10B). In addition, E1 PCV2 reacts with DNA to form nearly 100% covalent adduct (FIG. 10B).

In one or more embodiments, an RNA-binding protein of the present disclosure includes the amino acid sequence of E2 PCV2 (sometimes referred to as “PCV2_E2,” “E2,” or SEQ ID NO:3). In one or more embodiments, an RNA-binding protein of the present disclosure includes an amino acid sequence having at least 70%, at least 75%, at least 80%, at least 85%, at least 90%, at least 95%, at least 98%, or at least 99% sequence identity with SEQ ID NO:3. As is described herein, E2 PCV2 is at least partially characterized by a sequence spanning from V80 to K86 or G77 to T88 (Motif B, SEQ ID NO:7). As described in the Examples of the present disclosure, E2 PCV2 exhibits increased reactivity with RNA relative to WT PCV2 (FIG. 10B). In addition, E2 PCV2 reacts with DNA to form nearly 100% covalent adduct (FIG. 10B).

In one or more embodiments, an RNA-binding protein of the present disclosure includes the amino acid sequence of E3 PCV2 (sometimes referred to as “PCV2_E3,” “E3,” SEQ ID NO:2). In one or more embodiments, an RNA-binding protein of the present disclosure includes an amino acid sequence having at least 70%, at least 75%, at least 80%, at least 85%, at least 90%, at least 95%, at least 98%, or at least 99% sequence identity with SEQ ID NO:2. As is described herein, E1 PCV2 is at least partially characterized by a sequence spanning from V80 to K86 or G77 to T88 (Motif C, SEQ ID NO:8). E3 PCV2 includes an additional motif mutated relative to WT PCV2 spanning from 148 to L52 (Motif D, SEQ ID NO:10).

The engineered variants of PCV2 E1, E2, and E3 share a common mutated region from G77 to T88, more particularly, from V80 to K86, generalized to the motif of SEQ ID NO:9. In one or more embodiments, an RNA-binding protein includes an amino acid sequence including SEQ ID NO:9. In one or more embodiments, an RNA-binding protein includes an amino acid sequence having at least 70%, at least 75%, at least 80%, at least 85%, at least 90%, at least 95%, at least 98%, or at least 99% sequence identity with SEQ ID NO:9.

The RNA-binding proteins of the present disclosure may be identified by their activities. In one or more embodiments, an RNA-binding protein of the present disclosure is able to bind RNA in mammalian cells. In one or more embodiments, an RNA-binding protein of the present disclosure is able to bind RNA in an isolated composition free of cells.

In one or more embodiments, a nucleic acid encoding an RNA-binding protein of the present disclosure may be provided.

### Fusion Proteins

In another aspect, the present disclosure describes a fusion protein including an RNA-binding protein. An RNA-binding protein may be provided as a fusion protein with a protein involved in gene editing. Proteins involved in gene editing include, for example, adenosine deaminases, cytidine deaminases, CRISPR associated proteins such as Cas9, Cpf1, Cas12, or Cas13, adenosine deaminases acting on RNA (ADAR) proteins.

In one or more embodiments, a fusion protein may include a peptide or protein for affinity purification of the fusion protein, such as, for example, a 6×His-tag, a SUMO tag, a FLAG tag, or a hemagglutinin tag. In one or more embodiments, a fusion protein may include a detectable label such as a fluorescent protein. In one or more embodiments, a fusion protein may include proteins involved in eukaryotic mRNA stability or translation, such as, for example, the eukaryotic imitation factors EIF4A or EIF1, or the eukaryotic poly-A binding protein PABPC1.

In one of more embodiments, an RNA-binding protein may be provided as a fusion protein with a binding protein for the delivery of bound RNAs to cells. Binding proteins include nanobodies, FN3 domains, peptides, or protein G.

### Compositions

In another aspect, the present disclosure describes compositions including a protein. The composition may include an RNA-binding protein. The composition may include a fusion protein as described herein.

In one or more embodiments, a composition includes a metal salt, such as a magnesium salt or a manganese salt. Suitable metal salts include, for example, magnesium (II) chloride (MgCl₂), magnesium sulfate heptahydrate (MgSO₄·7H₂O), manganese (II) chloride tetrahydrate (MnCl₂·4H₂O), and manganese (II) sulfate monohydrate (MgSO₄·H₂O). A composition may include a salt that dissolves in water to yield a divalent cation, such as Mg²⁺ and/or Mn²⁺. In one or more embodiments, a composition may include at least 50 μM, at least 100 μM, at least 200 μM, at least 500 μM, at least 700 μM, at least 1.0 mM, or at least 2.0 mM of a divalent metal salt. A composition may include other suitable salts, such as, for example, sodium chloride (NaCl).

In one or more embodiments, the composition may include a polynucleotide. As used herein, a polynucleotide includes two or more nucleotides. The polynucleotide may include RNA bases, DNA bases, or a combination thereof. The polynucleotide may be single-stranded or double stranded. The polynucleotide may include naturally occurring nucleotides and/or non-naturally occurring nucleotides.

In one or more embodiments, a polynucleotide of a composition may be a guide RNA, such as a single guide RNA (sgRNA) or a prime editing guide RNA (pegRNA). A polynucleotide may be another polynucleotide involved in gene editing, such as a repair template (ssODN).

In one or more embodiments, the composition may include a buffering component, such as 4-(2-hydroxyethyl)-1-piperazineethanesulfonic acid (HEPES), TrisHCl, 2-{[1,3-Dihydroxy-2-(hydroxymethyl)propan-2-yl]amino}ethane-1-sulfonic acid (TES), 2,2′-(Piperazine-1,4-diyl)di(ethane-1-sulfonic acid) PIPES, phosphate, or tricine. The composition may have a pH between 6.0 and 10.0, such as 7.0 to 9.0, such as 8.0. The composition may include any other suitable components such as bovine serum albumin, salmon sperm DNA, PEG, dimethyl sulfoxide (DMSO), formamide, or dithiothreitol.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be performed in any feasible order. And, as appropriate, any combination of two or more steps may be performed simultaneously.

As used herein, the word “exemplary” means to serve as an illustrative example and should not be construed as preferred or advantageous over other embodiments.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

### EXEMPLARY EMBODIMENTS

Embodiment 1 is an RNA-binding protein comprising a substitution mutation to a semi-conserved domain, said semi-conserved domain comprising the amino acid sequence of SEQ ID NO:5.

Embodiment 2 is the RNA-binding protein of Embodiment 1, wherein the RNA-binding protein comprises a catalytic tyrosine residue and one or two metal-coordinating histidine residues.

Embodiment 3 is the RNA-binding protein of any preceding Embodiment, wherein the RNA-binding protein is an HUH endonuclease.

Embodiment 4 is the RNA-binding protein of any preceding Embodiment, wherein the RNA-binding protein is virally derived.

Embodiment 5 is the RNA-binding protein of Embodiment 4, wherein the RNA-binding protein is derived from a virus belonging to the genus Circovirus.

Embodiment 6 is the RNA-binding protein of Embodiment 5, wherein the RNA-binding protein derived from a porcine circovirus (PCV).

Embodiment 7 is the RNA-binding protein of Embodiment 6, wherein the RNA-binding protein derived from porcine circovirus-2 (PCV2).

Embodiment 8 is the RNA-binding protein of any preceding Embodiment, wherein the RNA-binding protein comprises an amino acid sequence comprising at least 70% identity to SEQ ID NO:1.

Embodiment 9 is the RNA-binding protein of Embodiment 8, further comprising a substitution mutation to the position functionally equivalent to position 81 in SEQ ID NO:1 and/or a substitution mutation to the position functionally equivalent to position 86 in SEQ ID NO:1.

Embodiment 10 is the RNA-binding protein of Embodiment 9, wherein the substitution mutation to position 81 and/or the substitution mutation to position 86 comprises an aromatic amino acid.

Embodiment 11 is the RNA-binding protein of Embodiment 10, wherein the substitution mutation to position 81 comprises tryptophan.

Embodiment 12 is the RNA-binding protein of Embodiment 10 or 11, wherein the substitution mutation to position 86 comprises tyrosine.

Embodiment 13 is the RNA-binding protein of any preceding Embodiment, wherein the RNA-binding protein comprises the amino acid sequence of any one of SEQ ID NO:2, SEQ ID NO:3, or SEQ ID NO:4.

Embodiment 14 is the RNA-binding protein of any preceding Embodiment, wherein the RNA-binding protein comprises a divalent cation.

Embodiment 15 is the RNA-binding protein of Embodiment 14, wherein the divalent cation comprises Mg²⁺ or Mn²⁺.

Embodiment 16 is the RNA-binding protein of Embodiment 15, wherein the divalent cation comprises Mn²⁺.

Embodiment 17 is a nucleic acid expressing the RNA-binding protein of any preceding Embodiment.

Embodiment 18 is a composition comprising the RNA-binding protein of any preceding Embodiment.

Embodiment 19 is the composition of Embodiment 18, wherein the composition comprises Mn²⁺.

Embodiment 20 is a fusion protein comprising the RNA-binding protein of any preceding Embodiment.

Embodiment 21 is the fusion protein of Embodiment 20, wherein the fusion protein comprises a protein involved in gene editing.

Embodiment 22 is the fusion protein of Embodiment 20, wherein the fusion protein comprising an adenosine deaminase.

Embodiment 23 is the fusion protein of Embodiment 22, wherein the adenosine deaminase is an adenosine deaminase acting on RNA (ADAR).

Embodiment 24 is a composition comprising the fusion protein of any one of Embodiments 20 to 23 and a nucleic acid, wherein the nucleic acid comprises the on sequence of the RNA-binding protein.

Embodiment 25 is a composition comprising the RNA-binding protein of any one of Embodiments 1 to 16 and a nucleic acid, wherein the nucleic acid comprises the ori sequence of the RNA-binding protein.

### EXAMPLES

The present invention is illustrated by the following examples. It is to be understood that the particular examples, materials, amounts, and procedures are to be interpreted broadly in accordance with the scope and spirit of the invention as set forth herein.

TABLE 1
Reagents used in the Examples:

| Reagent | Source |
|---|---|
| 2 × CLONEAMP HiFi PCR Premix | Takara Bio USA, Inc., San Jose, CA |
| Mutagenic oligonucleotide ULTRAMERS | Integrated DNA Technologies, Inc., |
| | Coralville, Iowa. |
| EBY100 Saccharomyces cerevisiae | American Type Culture Collection, Manassas, |
| | VA |
| 2 × YPAD | |
| 20 g/L Bacto Yeast Extract | Thermo Fisher Scientific, Inc., Waltham, MA |
| 40 g/L Bacto Peptone | Thermo Fisher Scientific, Inc., Waltham, MA |
| 40 g/L D-Glucose | Thermo Fisher Scientific, Inc., Waltham, MA |
| 25 mg/L Kanamycin | Thermo Fisher Scientific, Inc., Waltham, MA |
| 1 × pen/strep | Thermo Fisher Scientific, Inc., Waltham, MA |
| PEG 3350 | Sigma-Aldrich, St. Louis, MO |
| Lithium acetate | Sigma-Aldrich, St. Louis, MO |
| Salmon sperm DNA | Sigma-Aldrich, St. Louis, MO |
| Selective media | |
| 6.7 g/L Yeast Nitrogen Base | Sigma-Aldrich, St. Louis, MO |
| 5.0 g/L Casamino acids | Thermo Fisher Scientific, Inc., Waltham, MA |
| 2.13 g/L MES | Thermo Fisher Scientific, Inc., Waltham, MA |
| 2% D-Glucose | Thermo Fisher Scientific, Inc., Waltham, MA |
| 25 mg/L Kanamycin | Thermo Fisher Scientific, Inc., Waltham, MA |
| 1 × pen/strep | Thermo Fisher Scientific, Inc., Waltham, MA |
| Glucose | Sigma-Aldrich, St. Louis, MO |
| Potassium chloride (KCl) | Sigma-Aldrich, St. Louis, MO |
| HEPES | Sigma-Aldrich, St. Louis, MO |
| Sodium chloride (NaCl) | Sigma-Aldrich, St. Louis, MO |
| EDTA | Sigma-Aldrich, St. Louis, MO |
| Hydrophilic streptavidin magnetic beads | New England Biolabs, Inc., Ipswich, MA |
| Manganese chloride (MnCl2) | Sigma-Aldrich, St. Louis, MO |
| Anti-Myc antibody, FITC labeled | ICL, Inc., Portland, OR |
| LB | Thermo Fisher Scientific, Inc., Waltham, MA |
| BL21(DE3) competent E. coli | Agilent Technologies, Inc., Santa Clara, CA |
| IPTG | Sigma-Aldrich, St. Louis, MO |
| Complete protease inhibitor | Pierce Chemical Co., Rockford, IL |
| Ni-NTA HISPUR agarose beads | Thermo Fisher Scientific, Inc., Waltham, MA |
| DTT | Sigma-Aldrich, St. Louis, MO |
| 6 × His-ULP1 | New England Biolabs, Inc., Ipswich, MA |
| Imidazole | Thermo Fisher Scientific, Inc., Waltham, MA |
| RPMI-1640 | Thermo Fisher Scientific, Inc., Waltham, MA |
| Fetal bovine serum | Thermo Fisher Scientific, Inc., Waltham, MA |
| Penicillin/streptomycin | Thermo Fisher Scientific, Inc., Waltham, MA |
| Lipofectamine 3000 | Thermo Fisher Scientific, Inc., Waltham, MA |
| 4-20% mini-PROTEAN TGX Stain-free gels | Bio-Rad Laboratories, Inc., Hercules, CA |

### Example 1: Recombinant Expression WT PCV2 and Reaction with RNA

In this Example, wild-type (WT) PCV2 was tested for ability to bind RNA.

WT PCV2 was recombinantly expressed in E. coli as previously described (see, e.g., U.S. Patent No. 10,717,733). An ssRNA substrate including the WT PCV2 on sequence was obtained. 3 μM of WT PCV2 (SEQ ID NO:1) was incubated with 30 μM of ssRNA, 1 mM of MnCl2, 50 mM of NaCl, and 50 mM of HEPES, pH 8.0 at 37° C. overnight. These conditions were selected as they were thought to be the optimal conditions for this reaction. Following incubation, a first aliquot of the reaction was treated with RNase. A second aliquot of the reaction was treated with DNase. A third aliquot was not treated with either DNase or RNase.

An aliquot of the unreacted WT PCV2 and each reaction aliquot was run on a stain-free SDS-PAGE gel and imaged (FIG. 1B). Approximately 20% of the WT PCV2 was determined to have an increased molecular weight, which was attributed to binding to the ssRNA substate. The reaction aliquot treated with RNase, but not the aliquot treated with DNase, showed loss of the higher molecular weight band. This was determined to indicate that the increased molecular weight band observed was WT PCV2 covalently bond to the ssRNA.

This Example demonstrated that WT PCV2 covalently bound to an ssRNA substrate. However, only about 20% of a preparation of WT PCV2 covalently bound to the ssRNA substrate. Thus, WT PCV2 reacted with RNA at a rate too low to be practically useful.

### Example 2: Preparing Amplified Mutagenic Libraries of PCV2 Variants

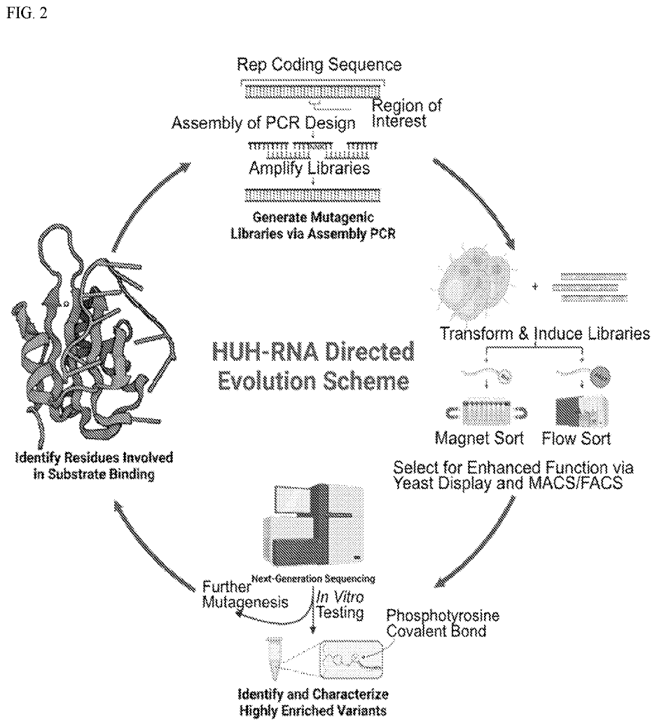

In this Example, a mutagenic library of WT PCV2 was created and amplified. A graphical schematic of this Examples 2-5 is shown in FIG. 2.

PCV2-RNA interaction engineering libraries were designed for site saturation mutagenesis of residues in proximity to ssDNA in co-crystal structures, starting with the single-stranded DNA bridging motif (sDBM), which is known to impart sequence specificity in Rep proteins, a subclass of HUH endonucleases. A maximum of six residues were mutagenized with “NNK” codons (which include all 20 amino acid possibilities plus a single stop codon) in a single library, producing a theoretical library size of 216, or roughly 85.8 million unique amino acid sequences. Alternative ambiguous codons, such as “DBK”, were occasionally used to limit residue variation to a subset of amino acids defined from the results of previous engineering stages. Residues showing strong convergence to a single amino acid at a given position would typically be excluded from further rounds of mutagenesis. Further library design was guided by in vitro testing of a panel of the most enriched variants from a previous round of selection when there was no strong convergence to specific residues or characteristics.

Mutagenic libraries were produced in a similar fashion as described by Lambert et al. 2020 (Lambert, Abigail R., Hallinan, Jazmine P., Werther, Rachel, Glow, Dawid, and Stoddard, Barry L., “Optimization of Protein Thermostability and Exploitation of Recognition Behavior to Engineer Altered Protein-DNA Recognition.” Structure 28:760-775). Briefly, insert libraries were designed with approximately 100 base pair overlaps with the 5′ and 3′ cleaved ends of the pETcon yeast surface display vector. Assembly PCR was used to generate the mutagenic inserts from a collection of oligonucleotides. The collection of oligonucleotides used for the assembly reaction was designed by the DNAWorks web server (Hoover, David M, and Jacek Lubkowski. “DNAWorks: an automated method for designing oligonucleotides for PCR-based gene synthesis.” Nucleic Acids Research vol. 30,10 (2002): e43. Doi:10.1093/nar/30.10.e43). Site saturation mutagenesis at the desired positions was achieved through the introduction of longer mutagenic oligonucleotide ultramers (Integrated DNA Technologies, Inc., Coralville, IA) in place of the shorter oligonucleotides spanning a region of interest.

Assembly inserts were produced through two sequential PCR reactions with 2×CLONEAMP HiFi PCR premix (Takara Bio USA, Inc., San Jose, CA). Both reactions were performed in 25 μL volumes, with the first reaction consisting of 1 μL of a 1 μM mixture of the coding region-spanning assembly oligonucleotides and the 2× master mix. The second reaction consisted of 2.5 μL of the first reaction (non-purified) as a template for amplification, 2.5 μL of a long forward primer, 2.5 μL of a long reverse primer, each primer having approximately 100 base pairs of homology to the pETcon vector. The second reaction was then separated by electrophoresis on a 1% agarose gel and purified via gel extraction. The purified mutagenic insert was then further amplified through numerous additional PCR reactions using the same forward- and reverse primers and purified via either PCR cleanup or gel extraction. Purified mutagenic insert products were pooled and stored at −20° C. for future use.

### Example 3: Expression of PCV2 Libraries in Yeast

In this Example, the amplified mutagenic libraries of Example 2 were assembled into plasmids and expressed in yeast.

The amplified mutagenic libraries were assembled into the pETcon plasmid using the yeast's natural ability to perform homologous recombination by transformation with a mixture containing cleaved vectors and inserts with approximately 100 base pairs of homology to its 5′ and 3′ ends. Mutagenic libraries were transformed into EBY100 Saccharomyces cerevisiae using lithium acetate. Briefly, the night prior to a transformation a small frozen aliquot of non-transformed EBY100 Saccharomyces cerevisiae yeast was cultured in 25 mL of nonselective growth media (2×YPAD). The next morning, this overnight culture was used to inoculate 100 mL of fresh 2×YPAD in a 500 mL baffled shake flask at 30° C. shaking at 250 RPM until the culture reached a density of around 100 million cells/mL. 2.5 billion cells (approximately 25 mL) were then transferred to a 50 mL conical tube, spun down for five minutes at 3500×g, and then resuspended in sterile water. Resuspended cultures were spun down a second time and resuspended in a transformation mixture composed of 2.4 mL of 50% PEG 3350, 360 μL of 1M lithium acetate, 500 μL of denatured salmon sperm DNA, and a 340 μL mixture of approximately 20 μg of the cleaved pETcon yeast surface display plasmid and 40 μg of mutagenic insert. Cells resuspended in the transformation mixture were then transferred to a 15 mL culture tube and incubated in a water bath at 42° C. for 35 minutes, gently mixing every five minutes. After incubation in the water bath, the culture was transferred to a 50 mL conical containing a 25 mL of a 50:50 mixture of selective media and 20% w/v glucose solution for recovery. Following this, the recovery mixture was added to 500 mL of selective media with glucose in a 2 μL baffled shake flask and shaken at 250 RPM at 30° C. overnight.

The following morning, yeast cells equal to roughly 50-times the theoretical diversity of the library were washed in water and passaged into fresh selective media with raffinose and shaken at 250 RPM at 30° C. for culture prior to induction. Once the culture reached a density of roughly 90 million to 120 million cells per mL, the desired number of cells are again washed in water and transferred to selective media with galactose at a density of roughly 30 million cells/mL and left at room temperature overnight for induction of cell surface display.

### Example 4: Sorting Yeast to Identify Highly-Expressed PCV2 Variants

In this Example, the induced yeast cultures of Example 3 were sorted to identify highly expressed PCV2 variants.

After culture overnight, each yeast culture was subjected to two rounds of magnet-activated cell sorting (MACS) using a 3′ biotinylated substrate (SEQ ID NO:12) and magnetic streptavidin beads for functional enrichment. A final round of fluorescence-activated cell sorting (FACS) with 3′ Alexa Fluor 647-labeled substrates (SEQ ID NO:12) gated to sort out highly functional variants. The nucleic acid substrates used for selection was an RNA version of a simplified on with four and three DNA bases appended to the 5′ and 3′ ends, respectively, in order to enhance the stability of an otherwise unmodified RNA substrate.

For the initial round of MACS selection, 1.5 billion induced yeast cells were washed two times with a washing buffer (500 mM KCl, 10 mM NaCl, 50 mM HEPES, pH 7.5) and resuspended at a density of 100 million cells per mL in a reaction buffer (50 mM NaCl, 50 mM HEPES, pH 8.0, 1 mM MnCl₂, and 100 nM of a 3′ biotinylated selection substrate). Cells were incubated for 15 minutes at 37° C. Following incubation, cells were washed twice in washing buffer and resuspended at 100 million cells per mL in a bead-binding buffer (20 mM Tris, pH 7.5, 500 mM NaCl, 1 mM EDTA) and 1 mg of hydrophilic streptavidin magnetic beads (New England Biolabs, Inc., Ipswich, MA) equilibrated into the same buffer. Cultures were incubated with magnetic beads at 4° C. for two hours. After binding, unbound variants were removed by collecting the magnetic beads using a magnetic rack and removing the supernatant. Magnetic beads were washed twice with bead-binding buffer and resuspended in 5 mL of selective media with glucose for subsequent growth for 16 hours.

After 16 hours of growth, the cultures were spun down and resuspended with 1 mL of selective media with raffinose. Each culture was incubated on a magnetic rack at room temperature for five minutes to collect the magnetic beads and the non-bound supernatant was transferred to fresh selective media with raffinose for further growth and induction.

Following growth and induction, a second round of MACS was performed in a similar manner but the cells were incubated in reaction buffer with 50 nM of biotinylated selection substrate for five minutes. These conditions were selected to be more stringent than the first round of substrate incubation in order to identify higher-performing variants. Following incubation, cells were washed twice, resuspended, bound to hydrophilic streptavidin magnetic beads, incubated, selected, and cultured as described above.

For FACS selection, the desired number of induced yeast cells (typically approximately 100 million) were washed two times with the washing buffer and were then resuspended at 100 million/mL in a reaction buffer (50 mM NaCl, 50 mM HEPES, pH 8.0, 500 μM MnCl₂ and 50 nM Alexa Fluor 647-labeled substrate) for subsequent incubation and reaction at 37° C. After incubation, cells were washed twice in washing buffer and resuspended at 100 million cells/mL in a staining buffer (200 mM KCl, 10 mM NaCl, 50 mM HEPES, pH 7.5, 1:100 dilution of a FITC-labeled anti-Myc antibody) and then rocked in the cold room for up to two hours. Following this, cells were spun down and resuspended in this same staining buffer at roughly 50 million cells/mL and held on ice until sorted on a cell sorter (FACSARIA II P0287, Becton Dickenson & Co., Franklin Lakes, NJ). Flow-sorted cells were then transferred to fresh selective media with glucose and shaken overnight at 250 RPM at 30° C. prior to further culture of manipulation.

Plasmid DNA was extracted from the cultured FACS-selected cells using a ZYMOPREP yeast plasmid miniprep II kit (Zymo Research Corp., Irvine, CA), per the manufacturer's instructions. One milliliter of yeast cells at a density of roughly 100 million cells/mL were used for plasmid purification. Purified plasmids were then used as a template for a PCR amplification (Takara Bio USA, Inc., San Jose, CA) to both amplify the mutagenic sequences and append Next-Generation Sequencing adapters to both the 5′ and 3′ ends of the amplicons using extended primers (Integrated DNA Technologies, Inc., Coralville, IA). All samples were sent individually for Next-Generation Sequencing (AMPLICON-EZ; Genewiz, Inc., South Plainfield, NJ). Raw NGS reads were then processed using a custom python script that employs the Biopython package for parsing reads and translating them to amino acid sequences. Briefly, low quality reads were discarded, and sequences were then parsed and searched for a constant region 5′ of the mutagenized section. The mutagenized section was then translated into amino acids and each instance of a given sequence was counted. Amino acid sequences with the highest number of reads were used to select mutant variants for in vitro testing and to inform further library design.

This Example identified multiple variants of PCV2 that bound to RNA. The amino acid sequences of the three variants of interest are shown in FIG. 3C and SEQ ID NOs:2-4.

### Example 5: Expression and Testing of PCV2 Variants in E. coli

In this Example, each identified variant of interest from Example 4 was expressed in E. coli and purified. The RNA binding ability of each variant was characterized.

HUH endonuclease constructs were expressed in BL21(DE3) competent E. coli cells in a 1 L volume with LB broth. The growth temperature was reduced from 37° C. to 18° C. after the culture OD₆₀₀ reached 0.8 and cells were induced with 0.5 mM isopropyl β-d-1-thiogalactopyranoside (IPTG) and incubated overnight. Cells were lysed in a lysis buffer (250 mM NaCl, 50 mM HEPES, pH 8.0, 1 mM EDTA) with a complete protease inhibitor tablet and pulse sonicated at 4° C. Clarified supernatant was batch bound to Ni-NTA HISPUR agarose beads for one hour, loaded onto a gravity column. The column was then washed with 30 column volumes of wash buffer (250 mM NaCl, 50 mM HEPES, pH 8.0, 1 mM EDTA, 30 mM imidazole), and finally eluted with elution buffer (250 mM NaCl, 50 mM HEPES, pH 8.0, 1 mM EDTA, 300 mM imidazole). Purification efficacy was assessed via SDS-PAGE gel analysis and protein-containing fractions were dialyzed (250 mM NaCl, 50 mM HEPES, pH 8.0, 1 mM EDTA, 1 mM dithiothreitol) overnight at 4° C. Protein was further purified and buffer exchanged using the SUPERDEX 300 Increase 10/300 GL (GE Healthcare Technologies, Inc., Chicago, IL) size exclusion column into the lysis buffer for storage and characterization. For the production of SUMO-free protein, approximately 30 μg 6×His-ULP1 (a SUMO-specific protease) per liter of culture was added to the protein samples prior to overnight dialysis. Following this, protein samples were then batch-bound a second time with Ni-NTA HISPUR agarose beads (Thermo Fisher Scientific, Inc., Waltham, MA) to remove cleaved 6×His-SUMO and 6×His-ULP1 for subsequent buffer exchange as described above.

### In Vitro HUH Cleavage Reactions

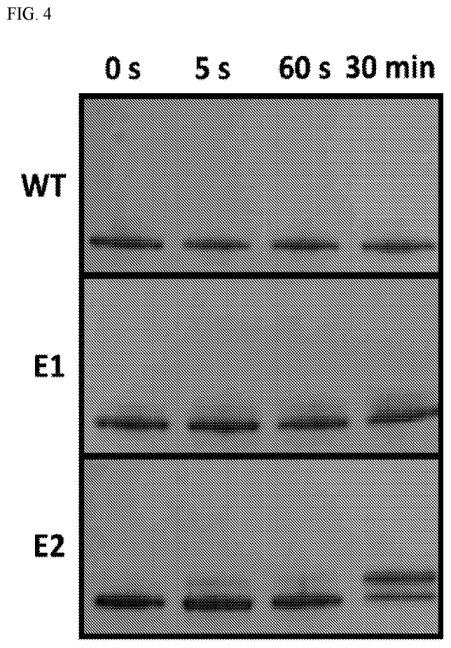

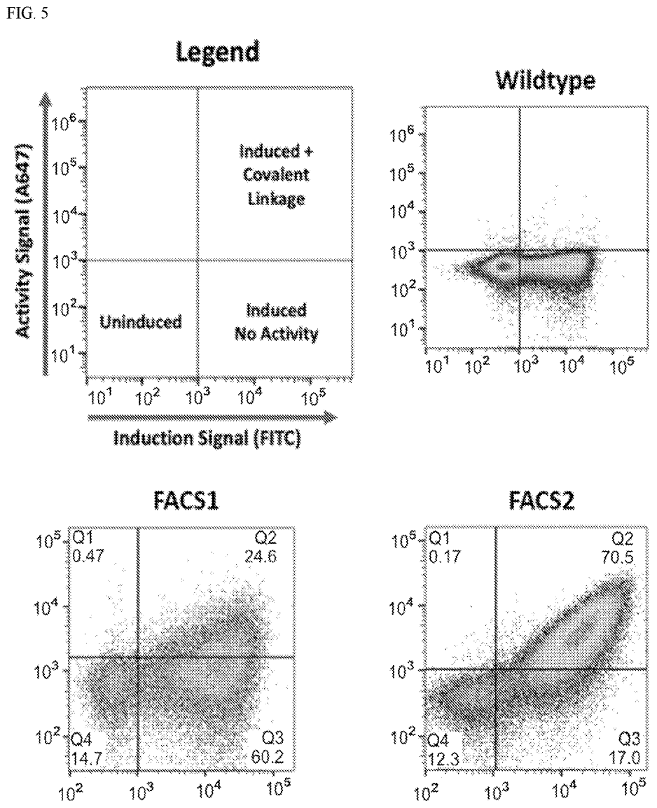

In vitro oligonucleotide cleavage reactions were carried out using final concentrations of 3 μM of each PCV2 variant and 30 μM single-stranded nucleic acid ori substrate in a cleavage buffer (50 mM NaCl, 50 mM HEPES, pH 8.0, 1 mM dithiothreitol, and 1 mM MnCl₂). Alternatively, restrictive reactions were carried out using final concentrations of 3 μM N-terminal SUMO-tagged PCV2 variant and 15 μM single-stranded nucleic acid ori substrate in a cleavage buffer of 50 mM NaCl, 50 mM HEPES, pH 8.0, 1 mM DTT, and 50 μM MnCl₂. Reactions were incubated at 37° C. before subsequent denaturation and SDS-PAGE analysis. Results from incubations with 30 μM single-stranded nucleic acid or substrate are shown in FIG. 5B. Results from incubations with 15 μM single-stranded nucleic acid on substrate are shown in FIG. 4 and FIG. 10B.

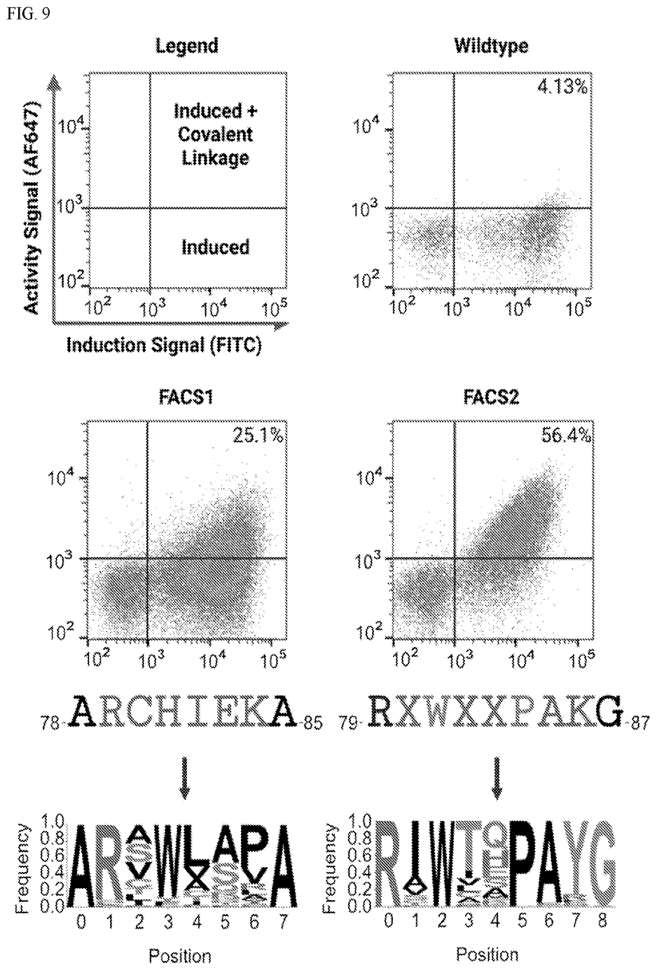

In vitro oligonucleotide cleavage reactions on the surface of yeast were carried out using final concentrations of 100 nM single-stranded nucleic acid ori RNA substrate labeled with AF647 in a cleavage buffer (50 mM NaCl, 50 mM HEPES, pH 8.0, and MnCl₂). Reactions were incubated at 37° C. for 15 minutes before subsequent fluorescent labeling of the C-terminal Myc-tag on the displayed proteins for analysis via flow cytometry. Reactions were carried out on a monoclonal population of yeast expression WT PCV2, or heterogenous populations of yeast expression mutagenic libraries post-flow sorting rounds FACS1 or FACS2. Independent Replicate results are shown in FIG. 5A and FIG. 9.

Variants E1 and E2 were able to bind RNA, with variant E2 binding RNA at a higher rate than E1.

### Molecular Beacon Cleavage Assays

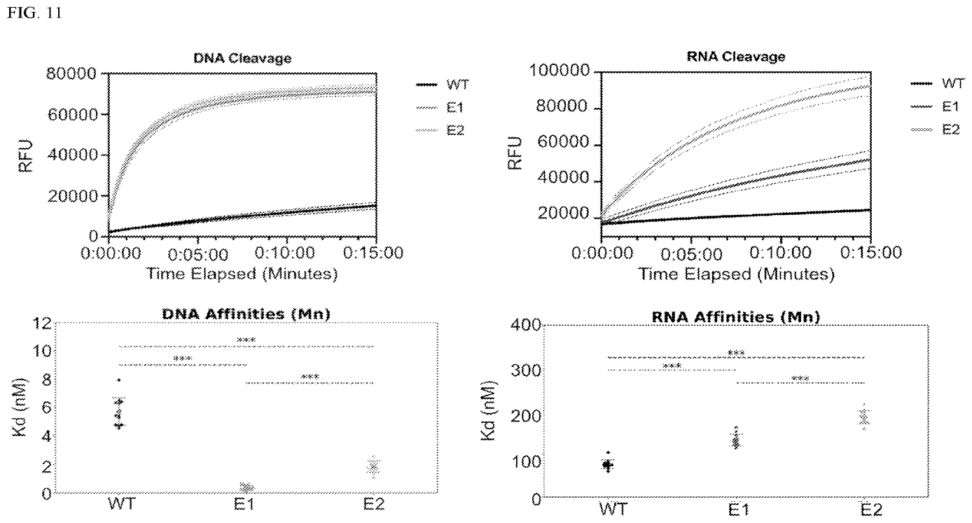

The cleavage rate of engineered and WT HUH-endonuclease variants was evaluated with a molecular beacon assay using a ssRNA probe harboring an ori-derived sequence labeled with a quencher/fluorophore (Q/F) substrate including SEQ ID NO:11 with a 5′ IOWA BLACK-FQ quencher and a 3′ FAM (Integrated DNA Technologies, Inc., Coralville, IA). All cleavage reactions were performed in a cleavage buffer of 50 mM NaCl, 50 mM HEPES, pH 8.0, 0.05% Tween-20, 1 mM DTT, and 50 μM MnCl₂. Purified HUH endonuclease variants were diluted with the cleavage buffer to 2 μM and the Q/F substrate was diluted to 200 nM in an identical buffer. In a black 96-well plate, 100 μL of the Q/F substrate was added to each well and inserted into a hybrid multi-mode plate reader (SYNERGY H1, Agilent Technologies, Inc., Santa Clara, CA). Using the injector module, 100 μL of a PCV2 variant was added to each well, initiating the reaction. Fluorescent signal was collected by the plate reader using the monochromator function with an excitation wavelength of 485±20 nm and emission collected using a 528±20 nm bandpass filter at room temperature. Emission values for all eight reactions were collected every five seconds for 10 minutes. Results of this assay are shown in FIG. 3A. In addition, E1 and E2 cleaved DNA more rapidly and more efficiently than WT PCV2 (FIG. 11). Further, E1 cleaved RNA somewhat more rapidly and efficiently than WT PCV2, but E2 cleaved RNA more efficiently and rapidly than WT PCV2 (FIG. 11).

This Example demonstrates that the RNA cleavage rate increased from each successive round of engineering, further corroborating previous findings that E1 PCV2 cleaves RNA more effectively than the WT PCV2, E2 PCV2 cleaves RNA more effectively than E1 PCV2, and E3 PCV2 cleaves RNA more effectively than E2 PCV2, E1 PCV2, and WT PCV2.

### Example 6: Testing Mammalian Activity of Engineered PCV2 Variants

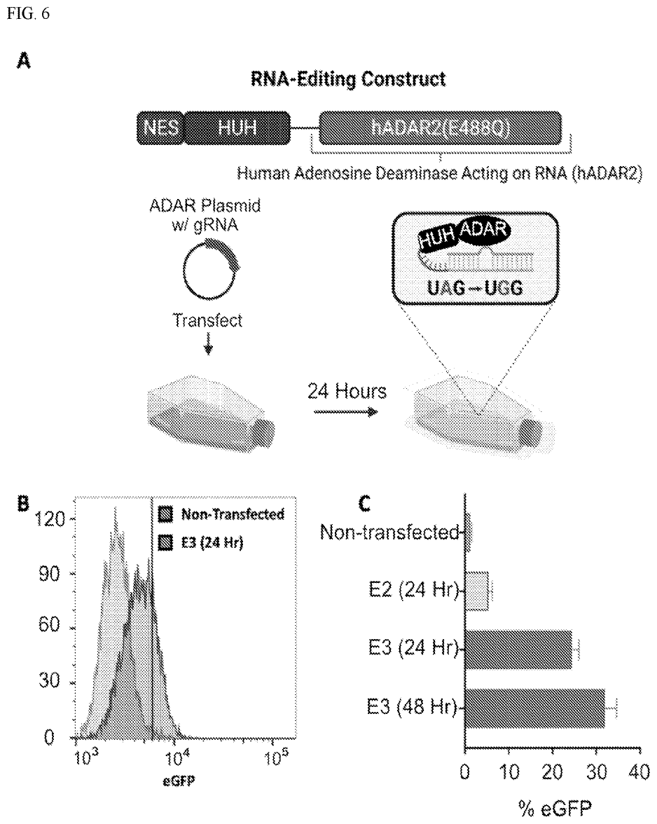

In this Example, the PCV2 variants E2 and E3 were tested for activity in mammalian cells. An Adenosine Deaminase Acting on RNA (ADAR)-HUH endonuclease fusion protein was used to edit an RNA transcript. The RNA transcript included an on sequence that was bound by the HUH endonuclease, localizing the ADAR to the RNA transcript. The ADAR then edited a “UAG” stop codon to a “UGG” tryptophan codon, resulting in eGFP expression in a K562 reporter cell line. A graphical representation of this Example is shown in FIG. 6A.

The cells used in this Example were K562 base-editing reporter cells that expressed a mutant eGFP construct with a tryptophan codon mutated to a stop codon. Without intervention, this reporter cell line did not express eGFP. Cells were regularly passaged every two to three days when high densities were achieved. Cells were maintained at 37° C. with 5% carbon dioxide in RPMI-1640 media supplemented with 10% fetal bovine serum and penicillin/streptomycin.

Cells were transfected with a plasmid encoding a CMV-driven E3-ADAR or E2-ADAR fusion protein and an RNA transcript complementary to the mutant eGFP transcript expressed by the reporter cells. The RNA transcript also included an HUH or sequence upstream of the complementary sequence. Cells were transfected by centrifugation at 500×g for five minutes, followed by aspiration of the supernatant and resuspension in transfection solutions containing Lipofectamine 3000 (Life Technologies Corp., Carlsbad, CA) and the pertinent plasmid. Cells were transfected in 24 well plates, each with 0.5 L of plasmid, 1.5 μL of Lipofectamine 3000, and 1 μL of the P3000 reagent, per manufacturer's instructions. Cells were then incubated for 24 to 48 hours and analyzed via flow cytometry. Results are shown in FIG. 6B and FIG. 6C.

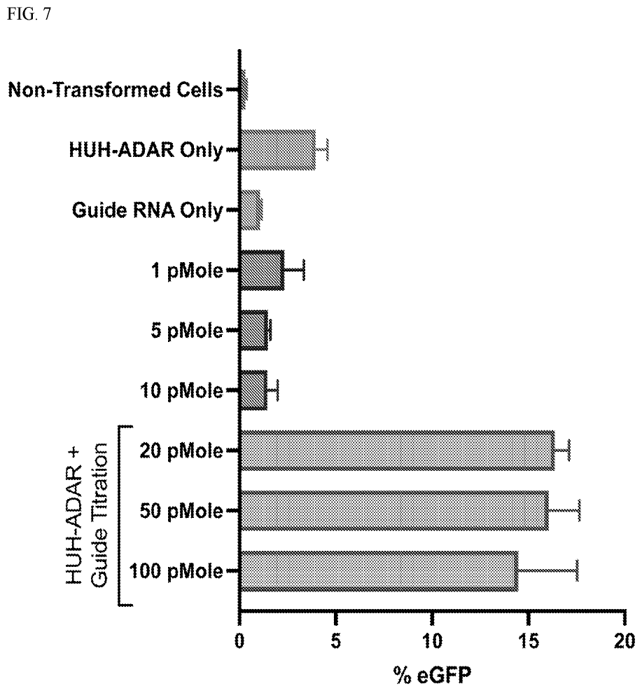

Cells transfected with the E3-ADAR fusion protein and the RNA transcript expressed eGFP (FIG. 6B, FIG. 6C). Specifically, over 20% of the transfected cells exhibited green fluorescence after a 24-hour treatment, and over 30% of the transfected cells exhibited green fluorescence after a 48-hour treatment. A titration of different guide concentrations demonstrated that 20 picomoles, 50 picomoles, or 100 picomoles of guide all yielded similar levels of editing (FIG. 7). This indicated that the E3-ADAR fusion bound the or sequence of the RNA transcript to form an E3-ADAR-RNA complex. This complex then bound to the mutant eGFP transcript, where the ADAR protein deaminated the adenosine of the UAG codon to result in a UGG codon. The modified eGFP transcript then was translated into a functional eGFP protein, which resulted in green fluorescence. Cells transfected with the E2-ADAR fusion protein and the RNA transcript expressed a low level of GFP, indicating that E2 was able to bind RNA in cells, but to a lower degree than E3.

Thus, an E2-ADAR or E3-ADAR fusion protein was used to edit mRNA in a mammalian cell.

### Example 7: Fluorescence Anisotropy Assays

The affinity of engineered PCV2 variants and WT PCV2 for ssDNA and ssRNA were measured via fluorescence anisotropy assays. 3′ fluorescein (FAM) labeled nucleic acid on substrates were used in all anisotropy experiments. Catalytically inactive PCV2 variants were titrated via 1:2 serial dilution starting at 1 μM into a 10 nM solution of a FAM-labeled substrate in a binding buffer of 50 mM NaCl, 50 mM HEPES, pH 8.0, 0.05% Tween-20, 1 mM DTT, and either 5 μM MnCl₂ or 1 mM EDTA. A single binding reaction was assembled in quadruplicate using four rows across a 96-well plate and three independent replicates of this reaction were performed across separate days. Reactions were incubated for 30 minutes at room temperature to ensure equilibrium and centrifuged at 2,000×g for two minutes before measurement. Mean dissociation constants (K_{D}) were calculated across three separate reactions (n=12) using the equations described below.

To calculate anisotropy from parallel and perpendicular fluorescence intensity values, data were fit to the following equation:

$$Anisotropy = ( Ipar - Iper ) / ( Ipar + 2 Iper )$$

where Ipar equals parallel fluorescence intensity and Iper equals perpendicular fluorescence intensity.

To calculate K_{D} under non-saturating ligand conditions, data were fit to a quadratic model:

$$A = ( {A}_{max} - {A}_{min} ) ( 2 [ {D}_{t} ] ) ( ( [ {P}_{t} ] + [ {D}_{t} ] + {K}_{D} ) - √ ( {( [ {P}_{t} ] + [ {D}_{t} ] + {K}_{D} )}^{2} - 4 [ {P}_{t} ] [ {D}_{t} ] ) ) + {A}_{min} ,$$

where [Dₜ] was the total amount of labeled ssDNA, [Pₜ] was the total amount of protein, K_{D} was the dissociation constant, Aₘₐₓ was the anisotropy ceiling, and Aₘᵢₙ was the anisotropy floor.

All anisotropy data were collected using a Synergy Neo2 Hybrid Multi-Mode Plate Reader and the Green FP Filter Cube (Agilent Technologies, Inc., Santa Clara, CA) with an excitation wavelength of 485 nm (20 nm bandwidth). Emissions of 528 nm were measured at ambient temperature. Measurements were fit and plotted in GraphPad Prism according to the above equations. Results from fluorescent anisotropy assays are shown in FIG. 11 for DNA (bottom left) and RNA (bottom right). Fluorescent anisotropy assays indicate that engineering PCV2 had minimal effects on its ability to non-covalently bind to DNA, and modest deleterious effects on its ability to non-covalently bind to RNA.

### Example 8: Covalent Linkage Quantification of E2 PCV2 Including H81 and/or K86

The E2 PCV backbone included H81W and K86Y mutations. To determine whether these mutations were necessary for RNA cleavage, variants were created and expressed including H81, K86, or both H81 and K86 in the E2 PCV backbone.

In vitro oligonucleotide cleavage reactions were carried out using final concentrations of 3 μM N-terminal SUMO-tagged PCV2 variant and 30 μM single-stranded nucleic acid Ori substrate (Integrated DNA Technologies, Inc., Coralville, IA) in a cleavage buffer of 50 mM NaCl, 50 mM HEPES, pH 8.0, 1 mM DTT, and 50 μM of MnCl₂. Reactions were incubated at 37° C. before subsequent denaturation and SDS-PAGE analysis.

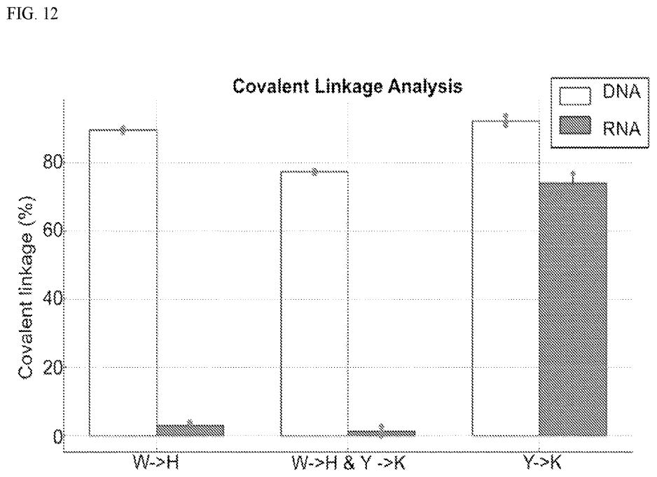

It was determined that H81W was necessary for cleavage of RNA, while K86W was not necessary for cleavage of RNA or DNA (FIG. 12).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. An RNA-binding protein comprising a substitution mutation to a semi-conserved domain, said semi-conserved domain comprising an amino acid sequence comprising SEQ ID NO:5.

2. The RNA-binding protein of claim 1, wherein the RNA-binding protein comprises a catalytic tyrosine residue and one or two metal-coordinating histidine residues.

3. The RNA-binding protein of claim 1, wherein the RNA-binding protein is an HUH endonuclease.

4. The RNA-binding protein of claim 1, wherein the RNA-binding protein is virally derived.

5. The RNA-binding protein of claim 4, wherein the RNA-binding protein is derived from a virus belonging to the genus Circovirus.

6. The RNA-binding protein of claim 5, wherein the RNA-binding protein derived from a porcine circovirus (PCV).

7. The RNA-binding protein of claim 6, wherein the RNA-binding protein derived from porcine circovirus-2 (PCV2).

8. The RNA-binding protein of claim 1, wherein the RNA-binding protein comprises an amino acid sequence comprising at least 70% identity to SEQ ID NO:1.

9. The RNA-binding protein of claim 8, further comprising a substitution mutation to the position functionally equivalent to position 81 in SEQ ID NO:1 and/or a substitution mutation to the position functionally equivalent to position 86 in SEQ ID NO:1.

10. The RNA-binding protein of claim 9, wherein the substitution mutation to position 81 and/or the substitution mutation to position 86 comprises an aromatic amino acid.

11. The RNA-binding protein of claim 10, wherein the substitution mutation to position 81 comprises tryptophan.

12. The RNA-binding protein of claim 11, wherein the substitution mutation to position 86 comprises tyrosine.

13. The RNA-binding protein of claim 1, wherein the RNA-binding protein comprises the amino acid sequence of any one of SEQ ID NO:2, SEQ ID NO:3, or SEQ ID NO:4.

14. The RNA-binding protein of claim 1, wherein the RNA-binding protein comprises a divalent cation.

15. The RNA-binding protein of claim 14, wherein the divalent cation comprises Mg²⁺ or Mn²⁺.

16. The RNA-binding protein of claim 15, wherein the divalent cation comprises Mn²⁺.

17. A nucleic acid expressing the RNA-binding protein of claim 1.

18. A composition comprising the RNA-binding protein of claim 1.

19. The composition of claim 18, wherein the composition comprises Mn²⁺.

20. A fusion protein comprising the RNA-binding protein of claim 1.

21. The fusion protein of claim 20, wherein the fusion protein comprises a protein involved in gene editing.

22. The fusion protein of claim 20, wherein the fusion protein comprises an adenosine deaminase.

23. The fusion protein of claim 22, wherein the adenosine deaminase is an adenosine deaminase acting on RNA (ADAR).

24. A composition comprising the fusion protein of claim 1 and a nucleic acid, wherein the nucleic acid comprises the on sequence of the RNA-binding protein.

25. A composition comprising the RNA-binding protein of claim 1 and a nucleic acid, wherein the nucleic acid comprises the on sequence of the RNA-binding protein.
